(19) United States
(12) Patent Application Publication
(10) Pub. No.: US 2025/0083145 A1
(43) Pub. Date: Mar. 13, 2025

(54) PIPETTE GUIDANCE IN MULTIPLE-WELL PLATE PATCH-CLAMP

(71) Applicant: SOPHION BIOSCIENCE A/S, Ballerup (DK)

(72) Inventors: Peder SKAFTE-PEDERSEN, Ballerup (DK); Kurt SOLGAARD, Graested (DK); Alec JORGENSEN, Ballerup (DK); Lasse HOMANN, Ballerup (DK); Jens HENNEKE, Ballerup (DK); Marie Louise Laub BUSK, Ballerup (DK)

(21) Appl. No.: 18/726,505

(22) PCT Filed: Jan. 9, 2023
(86) PCT No.: PCT/EP2023/050273

§ 371 Date: Jul. 3, 2024

(30) Foreign Application Priority Data
Jan. 7, 2022 (EP) 22150581.1

(51) Int. Cl. B01L 3/00; G01N 33/487

(52) U.S. Cl. CPC B01L 3/502761; G01N 33/48728; B01L 2200/0647; B01L 2300/0645; B01L 2300/087

(57) ABSTRACT

A patch-clamp system, and associated processes includes a multi-well patch-clamp plate and a manifold and can provide improved liquid control in patch-clamp experiments.

### TECHNICAL FIELD

A patch-clamp system, and associated processes, are provided. The invention provides improved liquid control in patch-clamp experiments carried out on a multi-well patch-clamp plate.

### BACKGROUND

Patch-clamp systems and methods are used to monitor electrophysiological properties of ion channels in ion channel-containing structures, typically lipid membrane-containing structures such as cells, by establishing an electrophysiological measuring configuration in which a cell membrane forms a high resistive seal around the measuring electrode, making it possible to determine and monitor a current flow through the cell membrane. Such systems can form part of an apparatus for carrying out patch-clamp techniques utilised to study ion transfer channels and biological membranes, for example.

The general idea of electrically insulating a patch of membrane and studying the ion channels in that patch under voltage-clamp conditions is outlined in Neher, Sakmann, and Steinback (1978) “The Extracellular Patch-Clamp, A Method For Resolving Currents Through Individual Open Channels In Biological Membranes”, Pflüger Arch. 375; 219-278. Recent developments in patch-clamp methodology have seen the introduction of planar substrates (e.g a silicon chip) in place of conventional glass micro pipette (for example, see WO 01/25769 and Mayer, 2000). Additional background art includes U.S. Pat. No. 8,268,260, which is hereby incorporated by reference.

A commercial instrument “QPatch” from Sophion Bioscience performs fully-automated patch-clamp measurements. The need exists, however, for more advanced liquid control than is possible with a fully-automated system. In particular, manual pipetting of liquids into a patch-clamp plate provides a greater flexibility to a user, but—on the other hand—patch-clamp systems typically require careful liquid addition to avoid excessive flow effects and to ensure even distribution of drugs to be tested. Additional complications may arise from variations in the pipetting techniques of individual users.

EP 2 681 550 describes a substrate for patch-clamp analysis. US2011/0251102 describes a multi-well plate and a patch clamp apparatus.

One object the invention is thus to provide a patch-clamp system, and associated processes, based on a multi-well patch-clamp plate, in which liquid control can be improved. In particular, the present technology allows pipetting of liquids into such a patch-clamp plate to be guided. Advantageously, the patch-clamp system has a simple construction and is straightforward to use. In particular, any moving parts in the system should be limited in their complexity.

### SUMMARY

A patch-clamp system is provided, said patch-clamp system comprising a patch-clamp plate and a manifold. The patch-clamp plate comprises: a plurality of intracellular (IC) inlets arranged in a first array, wherein each IC inlet is in fluid connection with an IC chamber; and a plurality of extracellular (EC) inlets arranged in a second array, wherein each EC inlet is in fluid connection with an EC chamber; a patch-clamp substrate arranged between each EC chamber and each IC chamber, each patch-clamp substrate comprising at least one patch-clamp hole, said patch-clamp hole extending through said patch-clamp substrate and providing a fluid connection between said EC and said IC chambers; wherein each IC chamber comprises an IC electrode; and each EC chamber comprises an EC electrode.

The manifold comprises a plurality of first pipetting channels arranged in a third array and optionally, a plurality of second pipetting channels arranged in a fourth array.

The manifold is moveable relative to the patch-clamp plate between a first position, in which each of the first pipetting channels, or each of the second pipetting channels, of said manifold are configured to align with one IC inlet of said patch-clamp plate, and a second position in which each of the first pipetting channels, or each of the second pipetting channels, of said manifold are configured to align with one EC inlet of said patch-clamp plate.

The system allows guided pipetting of liquids in the required positions in the plate, by moving the manifold between first and second positions.

A priming process, and a process for making patch-clamp measurements, are also provided. The invention further provides a patch-clamp instrument, comprising the patch-clamp system.

Further details of the technology are provided in the enclosed dependent claims, figures and examples.

### LEGENDS

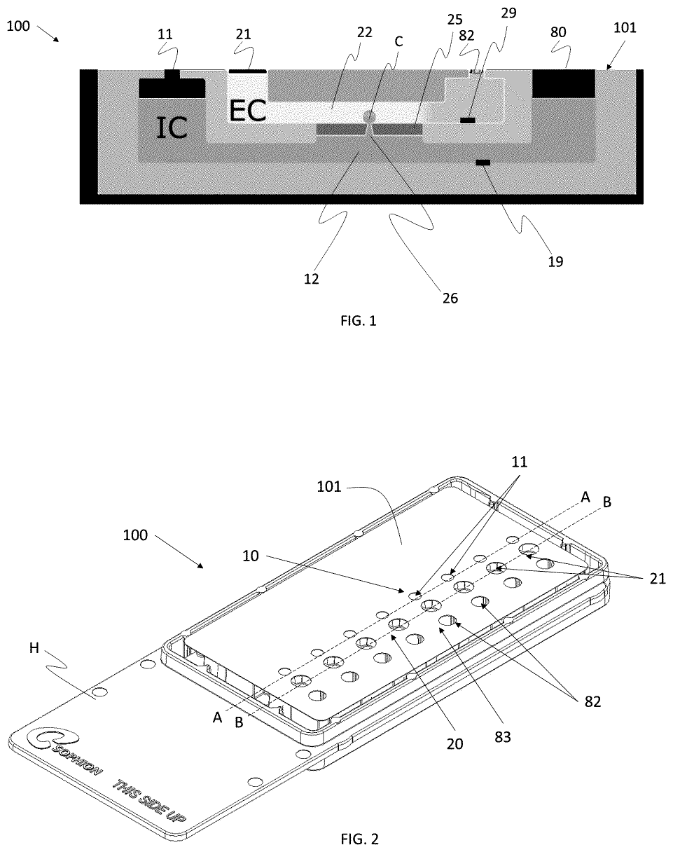

FIG. 1 is a schematic cross-sectional view of a patch-clamp plate according to the invention.

FIG. 2 shows a general, perspective view of a patch-clamp plate from above (i.e. the primary surface)

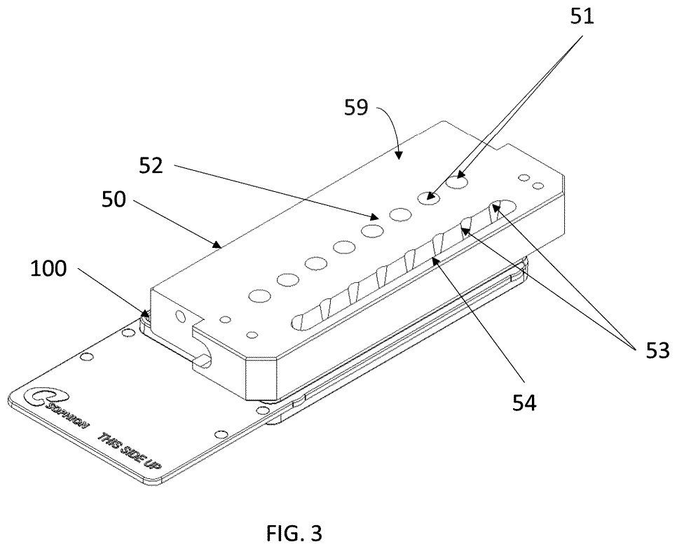

FIG. 3 shows a general perspective view of a patch-clamp plate, plus manifold, from above

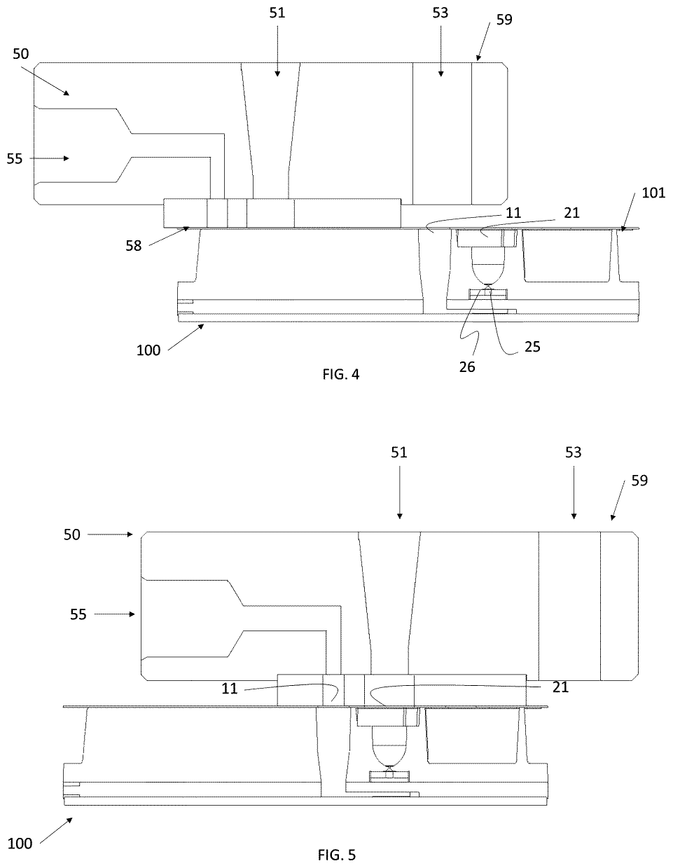

FIG. 4 shows a cross-sectional view of the patch-clamp plate and manifold, in a position ready for IC pipetting

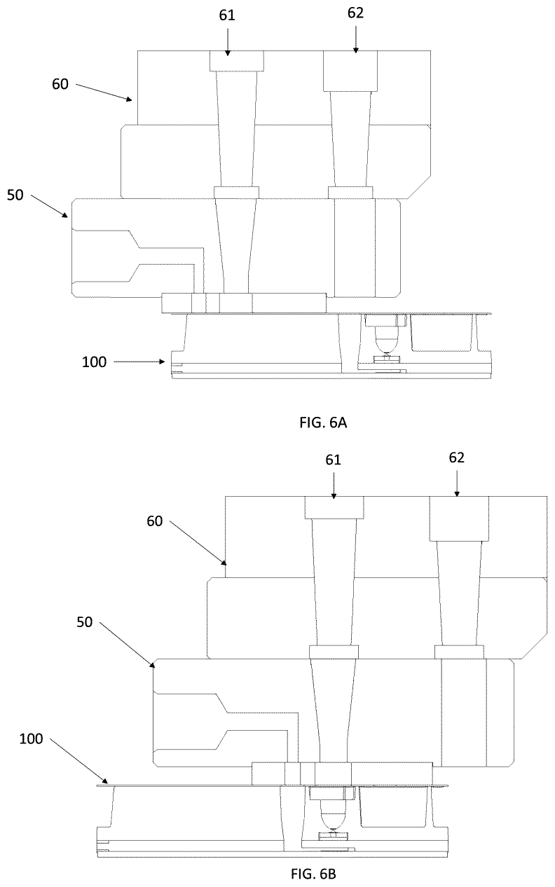

FIG. 5 shows a cross-sectional view of a patch-clamp plate and manifold, in a position ready for EC pipetting and pressure control

FIG. 6A shows a cross-sectional view of the patch-clamp plate, manifold and pipette guide, in a position ready for IC pipetting

FIG. 6B shows a cross-sectional view of a patch-clamp plate, manifold and pipette guide, in a position ready for EC pipetting and pressure control

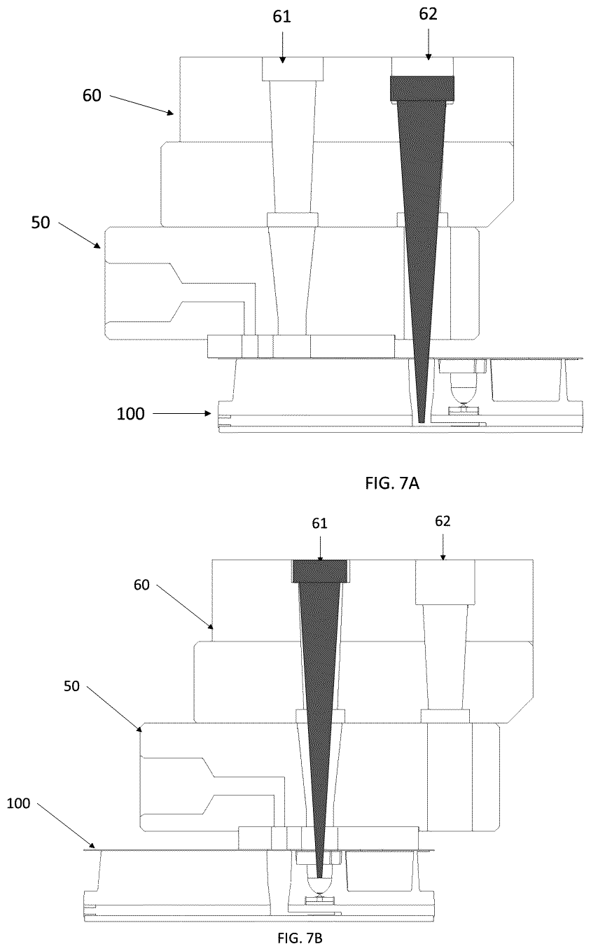

FIG. 7A shows a cross-sectional view of the patch-clamp plate, manifold and pipette guide, in a position ready for IC pipetting, as per FIG. 6A, with pipette tip in place.

FIG. 7B shows a cross-sectional view of a patch-clamp plate, manifold and pipette guide, in a position ready for EC pipetting and pressure control, as per FIG. 6B, with pipette tip in place.

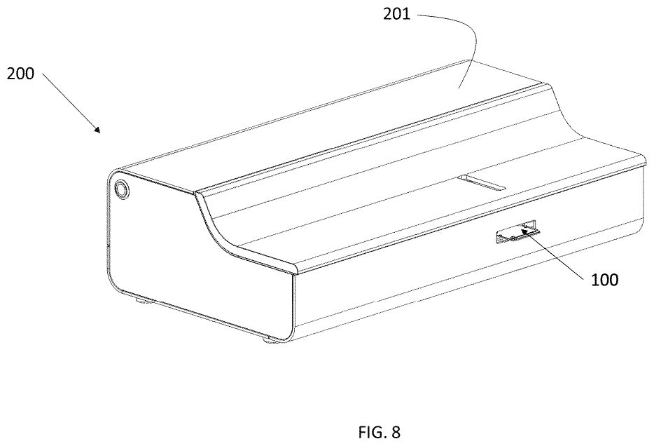

FIG. 8 shows a schematic view of a laboratory instrument, comprising the patch-clamp system of the invention.

### DETAILED DISCLOSURE

The patch-clamp technique is used to study ionic currents in individual isolated living cells, tissue sections, or patches of cell membrane. The technique is inter alia used in studies of neurons, cardiomyocytes, muscle fibres, and pancreatic beta cells.

Different patch-clamping techniques are used. In voltage clamp technique, the voltage across the cell membrane is controlled, and the resulting currents are recorded. In current clamp technique, the current passing across the membrane is controlled by the user and the resulting changes in voltage are recorded.

A patch-clamp system is provided, which comprises a patch-clamp plate and a manifold. Generally, the patch-clamp plate captures and holds cells while patch-clamp experiments are performed; the manifold controls air pressure and guides various pipetting procedures.

### Patch-Clamp Plate

The patch-clamp plate is used in automated patch-clamp (APC) measurements for high-throughput recordings of ion channel currents in living cells. The patch-clamp plate is assembled from several components with a range of properties that are advantageous for APC measurements.

The patch-clamp plate is typically “single-use”; i.e. it is manufactured in a simple manner from disposable materials (primarily moulded plastic), so that it can be disposed of after each use. In this manner, contamination between experiments can be reduced or completely eliminated.

The patch-clamp plate comprises a plurality of intracellular (IC) inlets arranged in a first array, wherein each IC inlet is in fluid connection with an IC chamber. The patch-clamp plate also comprises a plurality of extracellular (EC) inlets arranged in a second array, wherein each EC inlet is in fluid connection with an EC chamber.

The layout of the first and second arrays are suitably identical, so that neighbouring IC inlets in the first array are separated by the same distance in the same direction as neighbouring EC inlets in the second array.

Suitably, the plurality of IC inlets in the patch-clamp plate are arranged along a first axis (A-A) in a linear first array, and the plurality of EC inlets in the patch-clamp plate are arranged along a second axis (B-B) in a linear second array, and wherein—preferably—the first axis (A-A) and the second axis (B-B) are parallel. In this manner, alignment of the pipetting channels in the manifold with the EC/IC inlets in the plate is simplified.

The patch-clamp plate also comprises a patch-clamp chip (typically of silicon, but also optionally of plastic) arranged between each EC chamber and each IC chamber, each patch-clamp chip comprising one or more patch holes. The patch hole(s) extend(s) through the patch-clamp chip and providing a fluid connection between EC and said IC chambers. The biological cells to be tested are captured on the patch hole during patch-clamp experiments.

Each IC chamber of the patch-clamp plate comprises an IC electrode; and each EC chamber comprises an EC electrode.

The patch-clamp plate may additionally comprise a cover, which shields the testing environment and provides inlets for liquid access to the experiment site. A flexible gasket may also be present, to form the liquid- and electrical seal between the EC and IC chambers. The IC and EC electrodes (which are suitably Ag/AgCl electrodes) are typically embedded in a ceramic substrate. Once assembled, the components form two microfluidic channels placed above and below the patch site which contain the extra- and intracellular solutions respectively. The liquid inlets and outlets of the channels are placed away from the patch site to protect it from contamination.

The patch-clamp plate suitably has a primary surface, said primary surface comprising the plurality of intracellular (IC) inlets arranged in a first array, and the plurality of extracellular (EC) inlets arranged in a second array. When IC and EC inlets are arranged in the same surface of the patch-clamp plate, pipetting access is simplified, as it can take place from the same surface. Typically, in use, the primary surface of the patch-clamp plate is arranged to face upwards, and the manifold is arranged above this primary surface.

The glass surface at the experiment site enables the formation of a tight, giga-ohm (GΩ) seal between the cell membrane and patch hole using physiological solutions. This allows a user to avoid “seal-enhancing” ions, such as fluoride, which are often required in APC experiments and have been reported to undesirably modulate ion channel function.

The microfluidic channels connecting the extracellular liquid inlet to the patch site are suitably covered with a layer of glass, which minimizes compound adsorption. This ensures reliable compound concentrations in concentration-response experiments.

The microfluidic channel on the extracellular side allows rapid liquid exchange with multiple compound additions to the same cell during an experiment. The compound consumption is significantly reduced as solution exchange can be achieved with less than 10 μL of solution in the illustrated design. The laminar flow in the microfluidic channel prevents dilution of the compound during addition and subsequently allows complete compound washout. This eliminates the need for concentration compensation which might cause overshoot responses.

The patch-clamp plate typically contains 8 measurement sites. Each “measurement site” comprises the IC chamber, EC chamber and patch-clamp chip, with associated EC/IC inlets and outlets.

The embedded electrodes (IC or EC electrode) are suitably a single-use Ag/AgCl electrode. They ensure a high degree of measurement stability without the need for chlorination known from manual patch-clamping systems. The embedded electrodes in the patch-clamp plate allow stable measurements over time with minimum drift in voltage offset (dV_{off}/dt˜0.005 mV/min) and site resistance (dRₛᵢₜₑ/dt˜0.003 MΩ/min). The electrodes are arranged so that they can contact one or more electrical contacts in the patch-clamp system, whereby patch-clamp measurements can be taken.

The patch-clamp plate typically further comprises a plurality of IC pressure relief outlets arranged in a sixth array, wherein each IC pressure relief outlet is in fluid connection with an IC chamber, and a plurality of EC pressure relief outlets arranged in a seventh array, wherein each EC pressure relief outlet is in fluid connection with an EC chamber. The pressure relief outlets are open to the pressure system under use.

Each patch-clamp plate may additionally comprise a computer-readable code, e.g. barcode or QR code, to ensure traceability of the plate and the experiments.

A commercial patch-clamp plate suitable for use in the present technology is available from Sophion Bioscience A/S under the tradename “QPlate”.

The patch-clamp plate is preferably held within a holder (as illustrated most clearly in FIG. 2). The holder serves to manipulate the patch-clamp plate in a hygienic, safe manner, and holds it in the correct position within the patch-clamp system.

### Manifold

A manifold is arranged adjacent the patch-clamp plate in the patch-clamp system. The manifold can be used to selectively cover or uncover EC and IC inlets in the patch-clamp plate, and thereby guide various pipetting procedures. Suitably, the manifold can perform pressure protocols via the EC outlet and the IC inlet in the patch-clamp plate. Additionally, the relative alignment of the manifold relative to the patch-clamp plate can be automated.

The manifold is suitably formed from a single piece of metal (e.g. aluminium) with appropriate pipetting channels. The pipetting channels are suitably through-channels in the manifold, and are tapered from a wider opening distal from the patch-clamp plate to a narrower opening adjacent the patch-clamp plate, so as to follow the general tapered shape of a standard laboratory pipette tip. Each pipetting channel is designed to engage with (i.e. make contact with) a standard laboratory pipette tip, e.g. making contact with outer walls of the pipette tip. In this manner, secure and accurate positioning of the pipette tip can be achieved (suitably at least in the depth direction, but also in the plane of the patch-clamp plate).

Generally, the manifold comprises a plurality of first pipetting channels arranged in a third array. The third array of first pipetting channels is suitably identical to the layout of at least one of the first and third arrays of IC/EC inlets in the patch-clamp plate, so that—when the manifold and patch-clamp plate are superimposed—the first pipetting channels in the first array can align with the EC inlets in the second array and/or the IC inlets in the first array.

It may be sufficient that the manifold consists of only one array (i.e. the third array) of first pipetting channels. The movement of the manifold may thus be designed so that priming of the correct EC/IC liquids and cells via the required EC/IC inlets can be achieved.

Advantageously, the manifold may comprise a plurality of second pipetting channels arranged in a fourth array. The fourth array of second pipetting channels is suitably identical to the layout of at least one of the first and third arrays of IC/EC inlets in the patch-clamp plate. Use of a third and a fourth array of pipetting channels allows different liquids to be pipetted into the patch-clamp plate separately, and suitably at different depths in the patch-clamp plate.

The manifold is moveable relative to the patch-clamp plate. Suitably, the manifold is located adjacent the primary surface of the patch-clamp plate and is moveable relative to the patch-clamp plate at least in a plane parallel to said primary surface.

In particular, the manifold is moveable relative to the patch-clamp plate between a first position and a second position. In the first position, each of the first pipetting channels, or each of the second pipetting channels, of said manifold are configured to align with one IC inlet of said patch-clamp plate. In the first position, therefore, the manifold guides pipetting of IC liquids into the IC chamber via the IC inlets.

In the second position, each of the first pipetting channels, or each of the second pipetting channels of said manifold are configured to align with one EC inlet of said patch-clamp plate. In the second position, therefore, the manifold guides pipetting of EC liquids into the EC chamber via the EC inlets.

In a particular embodiment, the manifold comprises a plurality of first pipetting channels arranged in a third array, and a plurality of second pipetting channels arranged in a fourth array. In this embodiment—in the first position—each of the second pipetting channels of the manifold are configured to align with one IC inlet of said patch-clamp plate (100), and—in the second position—each of the first pipetting channels, of the manifold are configured to align with one EC inlet of the patch-clamp plate. As above, the use of a third and a fourth array of pipetting channels, arranged as above, allows different liquids to be pipetted into the patch-clamp plate separately, and suitably at different depths in the patch-clamp plate.

The patch-clamp system may further comprise a pipette guide, arranged such that the manifold is located between the pipette guide and said patch-clamp plate (i.e. on the side of the manifold opposite the patch-clamp plate). The pipette guide comprises a plurality of guide channels.

In particular, the pipette guide comprises a plurality of first guide channels, each of which is arranged to align with a first pipetting channel of the manifold, and, optionally, a plurality of second guide channels, each of which is arranged to align with a second pipetting channel of the manifold. Suitably, the pipette guide is temporarily or permanently fixed to the manifold, e.g. by screwing, welding, gluing or other suitable means.

The pipette guide provides improved possibilities for adjusting the alignment and/or depth of penetration of the pipette tip with respect to the patch-clamp plate. In particular, when temporarily fixed to the manifold, e.g. via screwing, adjustment becomes easier.

In the patch-clamp system presented herein, the manifold, optionally together with the pipette guide, may be configured to allow deeper penetration of a pipette tip into the patch-clamp plate via the plurality of second pipetting channels than the penetration of the same pipette tip into the patch-clamp plate via the plurality of first pipetting channels.

In one arrangement, the manifold comprises opposing first and second surfaces, wherein the first pipetting channels and the optional second pipetting channels extend through the manifold from said first surface to said second surface. In at least said first and said second positions, the first surface of the manifold is arranged to make contact with the primary surface of the patch-clamp plate. Suitably, in this arrangement, the manifold is clamped tightly against the patch-clamp plate, in the first and second positions. A gasket may be arranged to provide an airtight seal between the manifold and the patch-clamp plate.

Preferably, the first surface of the manifold does not make contact with the primary surface of the patch-clamp plate when the manifold is in a position between said first and second positions; i.e. it lifts away from the primary surface of the patch-clamp plate, so that movement between first and second positions is readily enabled, and the risk of damage to the manifold and/or the patch-clamp plate is reduced.

The manifold may further comprise one or more IC pressure channels arranged in a fifth array. The fifth array should have the same layout to the first array of IC inlets in the patch-clamp plate. In this manner, the IC pressure channels are arranged such that—in said second position—each of the IC pressure channels of said manifold are configured to align with each IC inlet in said first array. The IC pressure channels are used to (a) remove any trapped air in the IC chamber and (b) to provide a slight underpressure in the IC chamber, to fix the cell against the patch-clamp hole. Therefore, in the second position, each of the IC pressure channels of the manifold are configured to apply gas pressure or gas underpressure to each IC inlet in the first array.

The manifold may further comprise one or more EC pressure channels The EC pressure channels are arranged such that—in said second position—the EC pressure channels of said manifold are configured to align with each EC pressure relief outlet of the patch-clamp plate. The EC pressure channels are also used to (a) remove any trapped air in the EC chamber and (b) to provide a slight overpressure in the EC chamber, compared to the IC chamber to fix the cell against the patch-clamp hole. Therefore, in the second position, the EC pressure channels of the manifold are configured to apply gas pressure or gas underpressure to each EC pressure relief outlet.

When the manifold further comprises a plurality of pressure channels, the patch-clamp system may further comprise at least one pressure control device, being in fluid connection with each of the IC pressure channels and/or each of said EC pressure channels in the manifold and being configured to raise or lower the pressure in each of the IC pressure channels and/or each of said EC pressure channels of said manifold. In this manner, when the manifold is in the second position, the air pressure in the IC chamber and the EC chamber can be raised or lowered.

### Actuator

As noted above, the manifold and patch-clamp plate can be displaced relative to each other. The patch-clamp system suitably therefore comprises an actuator, which is preferably attached to the manifold, and which can move the manifold between said first and second positions. The actuator is arranged to actuate the manifold relative to the patch-clamp plate between said first position and said second position, and actuation preferably takes place at least in a plane parallel to the primary surface of the patch-clamp plate. The actuator may also be arranged to actuate the manifold in a second plane, being perpendicular to said primary surface, and preferably also being parallel with or including said first (A-A) and second (B-B) axes.

For simplicity, the actuator is preferably a linear actuator. The actuator is suitably arranged to clamp the first surface of the manifold against the primary surface of the patch-clamp plate in said first and said second positions, thereby providing a gas-tight seal.

The patch-clamp plate is suitably located in a holder (H), for ease of manipulation. The holder may be i a re-usable component which can be inserted into and withdrawn from the instrument.

### Priming

The manifold allows for individual pressure control on the intracellular side of the patch hole. This enables precise regulation of priming and whole-cell formation in an adaptive manner, which increases the experiment success rate. The priming protocol ensures the complete evacuation of air from the microfluidic channels before the beginning of the experiment

Cells are applied via the extracellular channel and positioned at the patch hole(s) by low, negative pressure across the patch hole(s). When properly sealed, a brief suction pulse brings the cell into the whole-cell configuration, where it is held in place by low, negative pressure throughout subsequent patch-clamp experiments. The extracellular solution can be exchanged throughout the experiment allowing precise addition and washout of compounds. The channel design allows solution exchange using volumes as low as 3 μL, thereby limiting compound consumption. The volume of the waste reservoir at the channel outlet, enables a total of ca. 250 μL solution being added in the course of the experiment.

A process for priming the patch-clamp system is thus provided. The process comprises the steps of:
providing the patch-clamp system as described herein,
with the manifold in the first position, pipetting IC liquid into each intracellular (IC) chamber of the patch-clamp plate via the IC inlet using the first pipetting channels in the manifold as a pipette guide;
moving the manifold to the second position, and
   pressurising each IC chamber via one or more IC pressure channels in the manifold, pipetting EC liquid into each extracellular (EC) chamber of the patch-clamp plate via the EC inlet using the first pipetting channels in the manifold as a pipette guide, and
   pressurising each EC chamber via one or more EC pressure channels in the manifold.

Suitably, the manifold is clamped against the patch-clamp plate in the second position.

After the priming process, cell capture may take place. This involves, with the manifold in the second position—pipetting biological cells (C) into each extracellular (EC) chamber of the patch-clamp plate via the EC inlet using the first pipetting channels in the manifold as a pipette guide, and then applying a negative pressure across the patch hole and thus capturing a biological cell at said patch hole.

### Patch-Clamp Measurements

As set out above, the patch-clamp system is used for performing patch-clamp measurements on biological cell(s).

A process is therefore provided for determining and/or monitoring the electrophysiological properties of ion channels in a biological cell, in the patch-clamp system defined herein, said process comprising the steps of:
providing the patch-clamp system as defined herein,
priming the patch-clamp system according to the priming process defined herein,
capturing one or more biological cells at said one or more patch hole, according to the process defined herein, and
performing electrophysiological measurements on said biological cell.

A patch-clamp instrument is also provided, suitable for a laboratory setting. The instrument comprises the patch-clamp system as defined herein, and also comprises:
a computer system having software, said computer system and associated software being arranged to:
   control the pressure control device,
   control the actuator,
   control one or more patch-clamp experimental parameters (e.g. cell stimulation),
   measure an electrical signal generated by a cell captured in the patch-clamp system, via said IC electrode and said EC electrode.

The instrument further comprises a user interface for communicating with a user of said instrument, said user interface being in electronic communication with said computer system. The user interface is suitably in the form of a graphical user interface, displayed on a computer display, preferably a touch-screen display.

Suitably, the instrument has a housing, in which said patch-clamp system and said computer system are arranged.

### DETAILED DESCRIPTION OF THE FIGURES

FIG. 1 is a schematic cross-sectional view of a patch-clamp plate 100 according to the invention. An intracellular (IC) inlet 11 is shown as being in fluid connection with an IC chamber 12 and an extracellular (EC) inlet 21 is in fluid connection with an EC chamber 22.

The IC and EC inlets open on the same (primary) face 101 of the plate 100. The EC chamber is arranged above the IC chamber (i.e. closer to the primary face 101). Various liquids can be pipetted into the EC/IC chambers via the respective EC/IC inlets.

A patch-clamp substrate 25 (in this figure, in the form of a chip) is arranged between the EC chamber 22 and the IC chamber 12. The patch-clamp substrate 25 comprises a patch-clamp hole 26, upon which cell (C) is captured and held in place by a slight underpressure in the IC chamber 12. The patch-clamp hole 26 extends through the patch-clamp substrate 25 and provides a fluid connection between said EC and said IC chambers 22, 12.

The IC chamber 12 comprises an IC electrode 19, while the EC chamber 22 comprises an EC electrode 29. Patch-clamp measurements on the cell C are essentially performed via measurement of current/voltage across the IC/EC electrodes.

FIG. 2 shows a general perspective view of a patch-clamp plate from above (i.e. the primary surface). Each IC/EC inlet and IC/EC chamber, together with the associated IC/EC electrodes and patch-clamp substrate 25, constitute a “site”. FIG. 2 shows that, in the patch-clamp plate, a plurality of intracellular (IC) inlets 11 are arranged in a first array 10, and a plurality of extracellular (EC) inlets 21 are arranged in a second array 20. In FIG. 2 the IC inlets 11 are arranged along first axis (A-A), while the EC inlets 21 are arranged along a second axis (B-B). FIG. 2 also shows holder H, in which the patch-clamp plate can be inserted and removed.

FIG. 3 shows a general perspective view of a patch-clamp plate, plus manifold, from above, showing their relative arrangements. As can be seen, manifold 50 covers the patch-clamp plate 100 at the primary surface 101 thereof. The manifold 50 has a plurality of first pipetting channels 51 arranged in a third array 52 and a plurality of second pipetting channels 53 arranged in a fourth array 54. First pipetting channels 51 and second pipetting channels 53 extend through the manifold 50 from the first surface 58 (not visible in FIG. 3) to the second surface 59 and the first surface 58 of said manifold 50 is arranged to make contact with the primary surface 101 of the patch-clamp plate 100. In FIG. 3, adjacent second pipetting channels overlap somewhat.

FIGS. 4-7 illustrate the functionality of the patch-clamp system; in particular the moveable function of the manifold 50 relative to the patch-clamp plate 100.

FIG. 4 shows the manifold 50 in a first position relative to the patch-clamp plate 100. It can be seen that—in this position—the second pipetting channels 53 are aligned with an IC inlet 11. IC liquids can thus be pipetted into the IC chamber.

In the second position (shown in FIG. 5), the first pipetting channel 51 are aligned with the EC inlet 21. Pipetting of EC liquids, and cells can take place into the EC chamber. At the same time, in this second position—the pressure channel 55 of the manifold 50 is aligned with the IC inlet 11. The manifold can be clamped in this position, forming a seal via the gasket. Various pressure protocols can be performed via the pressure channel, and—once a cell is captured at the patch hole—the system is ready to perform patch-clamp experiments.

FIGS. 6A and 6B are similar to FIGS. 4 and 5, and additionally show pipette guide 60 having first guide channels 61, and second guide channels 62. Adjustment of the pipette guide (or exchange of the pipette guide with a different pipette guide) allows pipetting at different depths and accommodates different pipette dimensions.

FIGS. 7A and 7B show how the manifold 50 and pipette guide 60 allow deeper penetration of a pipette tip into the patch-clamp plate 100 via the second pipetting channels 53 than the penetration of the same pipette tip into the patch-clamp plate 100 via the first pipetting channels 51. This helps to ensure proper pipette guidance and placement and optimum liquid addition to the liquid interface in the fluids of the EC and IC chambers.

FIG. 8 shows a schematic view of a laboratory instrument 200, comprising the patch-clamp system of the invention, showing housing 201 and where the patch-clamp plate 100 is located.

The present invention has been described with reference to a number of embodiments and figures. However, the skilled person is able to select and combine various embodiments within the scope of the invention, which is defined by the appended claims. All documents referenced herein are incorporated by reference.

### ASPECTS

The following list of numbered aspects is provided:

Aspect 1. patch-clamp system, said patch-clamp system comprising a patch-clamp plate (100) and a manifold (50),
wherein said patch-clamp plate (100) comprises:
   a plurality of intracellular (IC) inlets (11) arranged in a first array (10), wherein each IC inlet (11) is in fluid connection with an IC chamber (12); and
   a plurality of extracellular (EC) inlets (21) arranged in a second array (20), wherein each EC inlet (21) is in fluid connection with an EC chamber (22);
   a patch-clamp substrate (25) arranged between each EC chamber (22) and each IC chamber (12), each patch-clamp substrate (25) comprising at least one patch-clamp hole (26), said patch-clamp hole (26) extending through said patch-clamp substrate (25) and providing a fluid connection between said EC and said IC chambers (22, 12);
   wherein each IC chamber (12) comprises an IC electrode (19);
   and each EC chamber (22) comprises an EC electrode (29);
wherein said manifold (50) comprises
   a plurality of first pipetting channels (51) arranged in a third array (52),
   optionally, a plurality of second pipetting channels (53) arranged in a fourth array (54),
wherein the manifold (50) is moveable relative to the patch-clamp plate (100) between a first position, in which each of the first pipetting channels (51), or each of the second pipetting channels (53), of said manifold (50) are configured to align with one IC inlet (11) of said patch-clamp plate (100),
and a second position in which each of the first pipetting channels (51), or each of the second pipetting channels (53), of said manifold (50) are configured to align with one EC inlet (21) of said patch-clamp plate (100).

Aspect 2. The patch-clamp system according to aspect 1, wherein said manifold (50) comprises a plurality of first pipetting channels (51) arranged in a third array (52), and a plurality of second pipetting channels (53) arranged in a fourth array (54);
and wherein—in said first position—each of the second pipetting channels (53), of said manifold (50) are configured to align with one IC inlet (11) of said patch-clamp plate (100),
and wherein—in said second position—each of the first pipetting channels (51), of said manifold (50) are configured to align with one EC inlet (21) of said patch-clamp plate (100).

Aspect 3. The patch-clamp system according to any one of the preceding aspects, further comprising a pipette guide (60), arranged such that the manifold (50) is located between said pipette guide (60) and said patch-clamp plate (100), said pipette guide (60) comprising
a plurality of first guide channels (61), each of which is arranged to align with a first pipetting channel (51) of the manifold (50),
optionally, a plurality of second guide channels (62), each of which is arranged to align with a second pipetting channel (53) of the manifold (50).

Aspect 4. The patch-clamp system according to any one of aspects 2-3, wherein the manifold (50), optionally together with the pipette guide (60), is configured to allow deeper controlled penetration of a pipette tip into the patch-clamp plate (100) via the plurality of second pipetting channels (53) than the penetration of the same pipette tip into the patch-clamp plate (100) via the plurality of first pipetting channels (51).

Aspect 5. The patch-clamp system according to any one of the preceding aspects, wherein said patch-clamp plate (10) has a primary surface (101), said primary surface (101) comprising the plurality of intracellular (IC) inlets (11) arranged in a first array (10), and the plurality of extracellular (EC) inlets (21) arranged in a second array (20).

Aspect 6. The patch-clamp system according to aspect 5, wherein said manifold (50) is located adjacent the primary surface (101) of said patch-clamp plate (100) and is moveable relative to the patch-clamp plate (100) at least in a plane parallel to said primary surface (101).

Aspect 7. The patch-clamp system according to aspect 6, wherein said manifold (50) comprises opposing first (58) and second (59) surfaces, wherein the first pipetting channels (51) and the optional second pipetting channels (53) extend through the manifold (50) from said first surface (58) to said second surface (59), and wherein—in at least said first and said second position—the first surface (58) of said manifold (50) is arranged to make contact with the primary surface (101) of the patch-clamp plate (100).

Aspect 8. The patch-clamp system according to any one of the preceding aspects, wherein the plurality of IC inlets (11) in the patch-clamp plate (100) are arranged along a first axis (A-A) in a linear first array (10), and the plurality of EC inlets (21) in the patch-clamp plate (100) are arranged along a second axis (B-B) in a linear second array (20), and wherein—preferably—the first axis (A-A) and the second axis (B-B) are parallel.

Aspect 9. The patch-clamp system according to any one of the preceding aspects, further comprising an actuator (90) arranged to actuate the manifold (50) relative to the patch-clamp plate (10) between said first position and said second position, preferably wherein said actuation takes place at least in a plane parallel to said primary surface (101), more preferably wherein the actuator (90) is a linear actuator.

Aspect 10. The patch-clamp system according to aspect 9, wherein the actuator (90) is also arranged to actuate the manifold (50) in a second plane, being perpendicular to said primary surface (101), and preferably also being parallel with or including said first (A-A) and second (B-B) axes.

Aspect 11. The patch-clamp system according to any one of aspects 9-10, wherein the actuator (90) is arranged to clamp the first surface (58) of the manifold (50) against the primary surface (101) of the patch-clamp plate (10) in said first and said second positions.

Aspect 12. The patch-clamp system according to any one of the preceding aspects,
wherein said patch-clamp plate (100) further comprises a plurality of IC pressure relief outlets (80) arranged in a sixth array (81), wherein each IC pressure relief outlet (80) is in fluid connection with an IC chamber (12);
and
a plurality of EC pressure relief outlets (82) arranged in a seventh array (83), wherein each EC pressure relief outlet (82) is in fluid connection with an EC chamber (22).

Aspect 13. The patch-clamp system according to any one of the preceding aspects, wherein said manifold (50) further comprises one or more IC pressure channels (55) arranged in a fifth array (56); said IC pressure channels (55) being arranged such that—in said second position—each of the IC pressure channels (55) of said manifold (50) are configured to align with each IC inlet (11) in said first array (10).

Aspect 14. The patch-clamp system according to any one of the preceding aspects, wherein said manifold (50) further comprises one or more EC pressure channels; said EC pressure channels being arranged such that—in said second position—the EC pressure channels of said manifold (50) are configured to align with each EC pressure relief outlets (82).

Aspect 15. The patch-clamp system according to aspect 13-14, further comprising at least one pressure control device (70), said at least one pressure control device (70) being in fluid connection with each of the IC pressure channels (55) and/or each of said EC pressure channels in said manifold (50) and being configured to raise or lower the pressure in each of the IC pressure channels (55) and/or each of said EC pressure channels of said manifold (50).

Aspect 16. The patch-clamp system according to any one of the preceding aspects, further comprising a gasket arranged to provide an airtight seal between said manifold (50) and said patch-clamp plate (10).

Aspect 17. The patch-clamp system according to any one of the preceding aspects, wherein the patch-clamp substrate (25) is a silicon substrate or a polymer substrate.

Aspect 18. A process for priming the patch-clamp system according to any one of the preceding aspects, said process comprising the steps of:
providing the patch-clamp system according to any one of the preceding aspects,
with the manifold (50) in the first position, pipetting IC liquid into each intracellular (IC) chamber (12) of the patch-clamp plate (100) via the IC inlet (11) using the second pipetting channels (53) in the manifold as a pipette guide;
moving the manifold (50) to the second position, and
   pressurising each IC chamber via one or more IC pressure channels in the manifold;
   pipetting EC liquid into each extracellular (EC) chamber (22) of the patch-clamp plate (100) via the EC inlet (21) using the first pipetting channels (51) in the manifold as a pipette guide, and
   pressurising each EC chamber via one or more EC pressure channels in the manifold.

Aspect 19. A process for capturing one or more biological cells in a patch-clamp system according to any one of aspects 1-17, said process comprising the priming process according to aspect 18, and further comprising the steps of:
with the manifold (50) in the second position, pipetting biological cells (C) into each extracellular (EC) chamber (22) of the patch-clamp plate (100) via the EC inlet (21) using the first pipetting channels (51) in the manifold as a pipette guide,
applying a negative pressure across the patch hole (26) and thus capturing a biological cell at said patch hole (26).

Aspect 20. A process for determining and/or monitoring the electrophysiological properties of ion channels in a biological cell, in the patch-clamp system according to any one of the preceding aspects, said process comprising the steps of:
providing the patch-clamp system according to any one of aspects 1-17,
priming the patch-clamp system according to the process of aspect 18,
capturing one or more biological cells at said one or more patch hole (26), according to the process of aspect 19,
performing electrophysiological measurements on said biological cell.

Aspect 21. A patch-clamp instrument (200), said instrument (200) comprising the patch-clamp system according to any one of aspects 1-17, said instrument (200) comprising:
a computer system having software, said computer system and associated software being arranged to:
   control the pressure control device (70)
   control the actuator (90),
   control one or more patch-clamp experimental parameters,
   measure an electrical signal generated by a cell captured in the patch-clamp system, via said IC electrode (19) and said EC electrode (29),
said instrument (200) further comprising:
   a user interface for communicating with a user of said instrument, said user interface being in electronic communication with said computer system,
   optionally, a housing (201), in which said patch-clamp system and said computer system are arranged.

1.-15. (canceled)

16. A patch-clamp system, said patch-clamp system comprising a patch-clamp plate and a manifold,
wherein said patch-clamp plate comprises:
a plurality of intracellular (IC) inlets arranged in a first array, wherein each IC inlet is in fluid connection with an IC chamber; and
a plurality of extracellular (EC) inlets arranged in a second array, wherein each EC inlet is in fluid connection with an EC chamber;
a patch-clamp substrate arranged between each EC chamber and each IC chamber, each patch-clamp substrate comprising at least one patch-clamp hole, said patch-clamp hole extending through said patch-clamp substrate and providing a fluid connection between said EC and said IC chambers;
wherein each IC chamber comprises an IC electrode;
and each EC chamber comprises an EC electrode;
wherein said manifold comprises:
a plurality of first pipetting channels arranged in a third array,
optionally, a plurality of second pipetting channels arranged in a fourth array,
wherein the manifold is moveable relative to the patch-clamp plate between a first position, in which each of the first pipetting channels, or each of the second pipetting channels, of said manifold are configured to align with one IC inlet of said patch-clamp plate,
and a second position in which each of the first pipetting channels, or each of the second pipetting channels, of said manifold are configured to align with one EC inlet of said patch-clamp plate.

17. The patch-clamp system according to claim 16, wherein said manifold comprises a plurality of first pipetting channels arranged in a third array, and a plurality of second pipetting channels arranged in a fourth array; and
wherein—in said first position—each of the second pipetting channels, of said manifold are configured to align with one IC inlet of said patch-clamp plate, and
wherein—in said second position—each of the first pipetting channels, of said manifold are configured to align with one EC inlet of said patch-clamp plate.

18. The patch-clamp system according to claim 16, further comprising a pipette guide, arranged such that the manifold is located between said pipette guide and said patch-clamp plate, said pipette guide comprising:
a plurality of first guide channels, each of which is arranged to align with a first pipetting channel of the manifold,
optionally, a plurality of second guide channels, each of which is arranged to align with a second pipetting channel of the manifold.

19. The patch-clamp system according to claim 17, wherein the manifold, optionally together with the pipette guide, is configured to allow deeper controlled penetration of a pipette tip into the patch-clamp plate via the plurality of second pipetting channels than the penetration of the same pipette tip into the patch-clamp plate via the plurality of first pipetting channels.

20. The patch-clamp system according to claim 16, wherein said patch-clamp plate has a primary surface, said primary surface comprising the plurality of intracellular (IC) inlets arranged in a first array, and the plurality of extracellular (EC) inlets arranged in a second array, suitably, wherein said manifold is located adjacent the primary surface of said patch-clamp plate and is moveable relative to the patch-clamp plate at least in a plane parallel to said primary surface.

21. The patch-clamp system according to claim 16, further comprising an actuator arranged to actuate the manifold relative to the patch-clamp plate between said first position and said second position,
wherein said actuation takes place at least in a plane parallel to said primary surface,
wherein the actuator is a linear actuator.

22. The patch-clamp system according to claim 21, wherein the actuator is also arranged to actuate the manifold in a second plane, being perpendicular to said primary surface,
wherein the actuator is arranged to clamp the first surface of the manifold against the primary surface of the patch-clamp plate in said first and said second positions.

23. The patch-clamp system according to claim 16, wherein said patch-clamp plate further comprises a plurality of IC pressure relief outlets arranged in a sixth array,
wherein each IC pressure relief outlet is in fluid connection with an IC chamber; and
a plurality of EC pressure relief outlets arranged in a seventh array,
wherein each EC pressure relief outlet is in fluid connection with an EC chamber.

24. The patch-clamp system according to claim 16, wherein said manifold further comprises one or more IC pressure channels arranged in a fifth array; said IC pressure channels being arranged such that—in said second position—each of the IC pressure channels of said manifold are configured to align with each IC inlet in said first array.

25. The patch-clamp system according to claim 16, wherein said manifold further comprises one or more EC pressure channels; said EC pressure channels being arranged such that—in said second position—the EC pressure channels of said manifold are configured to align with each EC pressure relief outlets.

26. The patch-clamp system according to claim 24, further comprising at least one pressure control device, said at least one pressure control device being in fluid connection with each of the IC pressure channels and/or each of said EC pressure channels in said manifold and being configured to raise or lower the pressure in each of the IC pressure channels and/or each of said EC pressure channels of said manifold.

27. A process for priming the patch-clamp system according to claim 16, said process comprising the steps of:
providing the patch-clamp system,
with the manifold in the first position, pipetting IC liquid into each intracellular (IC) chamber of the patch-clamp plate via the IC inlet using the second pipetting channels in the manifold as a pipette guide;
moving the manifold to the second position, and
pressurising each IC chamber via one or more IC pressure channels in the manifold;
pipetting EC liquid into each extracellular (EC) chamber of the patch-clamp plate via the EC inlet using the first pipetting channels in the manifold as a pipette guide, and
pressurising each EC chamber via one or more EC pressure channels in the manifold.

28. A process for capturing one or more biological cells in a patch-clamp system according to claim 23, said process comprising the priming process, and further comprising the steps of:
with the manifold in the second position, pipetting at biological cells (C) into each extracellular (EC) chamber of the patch-clamp plate via the EC inlet using the first pipetting channels in the manifold as a pipette guide,
applying a negative pressure across the patch hole and thus capturing a biological cell at said patch hole.

29. A process for determining and/or monitoring the electrophysiological properties of ion channels in a biological cell, in the patch-clamp system according to claim 27, said process comprising the steps of:
providing the patch-clamp system,
priming the patch-clamp system,
capturing one or more biological cells at said one or more patch hole, according to the said further comprising the steps of with the manifold in the second position, pipetting at biological cells (C) into each extracellular (EC) chamber of the patch-clamp plate via the EC inlet using the first pipetting channels in the manifold as a pipette guide, applying a negative pressure across the patch hole and thus capturing a biological cell at said patch hole;
performing electrophysiological measurements on said biological cell.

30. A patch-clamp instrument, said instrument comprising the patch-clamp system according to claim 16, said instrument comprising:
a computer system having software, said computer system and associated software being arranged to:
control the pressure control device,
control the actuator,
control one or more patch-clamp experimental parameters,
measure an electrical signal generated by a cell captured in the patch-clamp system, via said IC electrode and said EC electrode,
said instrument further comprising:
a user interface for communicating with a user of said instrument, said user interface being in electronic communication with said computer system,
optionally, a housing, in which said patch-clamp system and said computer system are arranged.
